(19) United States
(12) Patent Application Publication
(10) Pub. No.: US 2015/0191742 A1
(43) Pub. Date: Jul. 9, 2015

(54) METHOD FOR ENHANCING RESISTANCE TO PATHOGENS IN PLANTS BASED ON OVER-EXPRESSION OF IMPAIRED OOMYCETE SUSCEPTIBILITY 1

(71) Applicant: NATIONAL TAIWAN UNIVERSITY, TAIPEI CITY (TW)

(72) Inventors: Laurent Zimmerli, Taipei City (TW); Ching-Wei Chen, Tainan City (TW); Yi-Ying Hsieh, New Taipei City (TW)

(73) Assignee: NATIONAL TAIWAN UNIVERSITY, TAIPEI CITY (TW)

(21) Appl. No.: 14/149,266

(22) Filed: Jan. 7, 2014

(51) Int. Cl. C12N 15/82

(52) U.S. Cl. CPC C12N 15/8279; C12N 15/8281; C12N 15/8282

(57) ABSTRACT

The present invention provides a method for enhancing resistance to pathogens in plants, comprising over-expression of Arabidopsis thaliana Impaired Oomycete Susceptibility 1 (IOS1) or homologs thereof in plants. The present invention also provides transgenic plants with enhanced resistance to pathogens by over-expression of Arabidopsis thaliana Impaired Oomycete Susceptibility 1 (IOS1) or homologs thereof in the plants.

### FIELD OF THE INVENTION

The present invention relates to a method for enhancing resistance to pathogens in plants based on over-expression of Impaired Oomycete Susceptibility 1 (IOS1).

### BACKGROUND OF THE INVENTION

Plants recognize microbial molecular signatures, collectively called pathogen/microbe-associated molecular patterns (PAMPs/MAMPs) by cell surface-localized pattern-recognition receptors (PRR). Recognition leads to the activation of a general, broad spectrum defense response called pattern-triggered immunity (PTI). Impaired Oomycete Susceptibility (IOS1) is a malectin-like leucine-rich repeat receptor-like kinase. Hok et al. (Hok S, Danchin E G, Allasia V, Panabières F, Attard A, Keller H. An Arabidopsis (malectin-like) leucine-rich repeat receptor-like kinase contributes to downy mildew disease. Plant Cell Environ. 2011 November; 34(11):1944-57) suggested a possible association between IOS1 and downy mildew disease, but these authors did not analyze the disease resistance ability of IOS1 over-expression lines.

### BRIEF DESCRIPTION OF THE DRAWINGS

FIG. 1. Arabidopsis thaliana transgenics over-expressing IOS1 are more resistant to Pst DC3000 and activate a primed PTI response.

(A) IOS1 mRNA expression levels in 3 IOS1 over-expression lines. Gene expression levels in 3 IOS1 over-expression lines OE1, OE2 and OE3 relative to wild-type Col-0 (defined value of 1) were analyzed by qRT-PCR. EF-1 and UBQ10 is used for normalization. Results are means±SD of 3 independent biological replicates each consisting of 3 technical repeats (n=9).
(B) Growth of Pst DC3000. Bacterial titers in 5-week-old Col-0 and IOS1 over-expression lines OE1, OE2 and OE3 were determined at 3 days post inoculation (dpi) with 1×10⁶ cfu/ml Pst DC3000. Values are means±SD of three independent biological replicates each with 3 technical repeats (n=9). Asterisks indicate a significant difference to Col-0 WT based on a t test (* P<0.05; ** P<0.01).
(C) Priming of callose deposition. Leaves of 5-week-old Col-0 WT and IOS1 over-expression lines OE1, OE2 and OE3 were syringe infiltrated with MgSO₄ (Mock) or 1 μM flg22 and samples were collected 6 h later for aniline blue staining. Numbers are averages±SD of number of callose deposits per square millimeter from 3 independent biological replicates each consisting of 9 technical repeats (n=27). Asterisks indicate a significant difference to WT Col-0 based on a t test (P<0.01).
(D) Priming of FRK1 expression. Leaves of 5-week-old Col-0 WT and IOS1 over-expression lines OE1, OE2 and OE3 were syringe infiltrated with MgSO₄ (Mock) or 1×10⁸ cfu/ml Pst DC3000 hrcC, and FRK1 expression levels were analyzed 90 mins later by qRT-PCR. EF-1 and UBQ10 are used for normalization. Relative gene expression levels are compared to mock-treated Col-0 WT (defined value of 1). Values are means±SD of three independent biological replicates each with 3 technical repeats (n=9). Asterisks indicate a significant difference to WT Col-0 based on a t test (P<0.01).
(E) MAPK activation is primed in lines over-expressing IOS1. Leaves of 5-week-old Col-0 and IOS1 over-expression lines OE1, OE2 and OE3 were infiltrated with 1 nM flg22 for 5 mins Immunoblot analysis using phospho-p44/42 MAP kinase antibody is shown in top panel. Arrowheads indicate the positions of MPK6 and MPK3. FastBlue-staining is used to estimate equal loading in each lane (bottom panel). This experiment is one of 2 independent replicates.
(F) Reinforced stomatal innate immunity. Stomatal apertures in leaf epidermal peels from 5-week-old Col-0 and IOS1 over-expression lines OE1, OE2 and OE3 were analyzed after 1.5 h or 3 h exposure to MgSO₄ (Mock) or 1×10⁸ cfu/ml Pst DC3000. Values are shown as means±SD of 3 independent replicates each consisting of at least 60 technical repeats (n>180 stomata). Asterisks indicate a significant difference to mock control based on a t test analysis (P<0.001).

FIG. 2. Arabidopsis thaliana transgenics over-expressing IOS1 are more resistant to infection of Botrytis cinerea. Arabidopsis Col-0 (wild-type) and over-expression lines OE1, OE2 and OE3 leaves were droplet-inoculated (10 μl) with 1×10⁵ B. cinerea spores/ml and B. cinerea disease symptoms were visualized 3 days later. Experiments were repeated 3 times with similar results.

FIG. 3. Nicotiana benthamiana transgenics over-expressing IOS1 (OE1 and OE2) are more resistant to infection of hemi-biotrophic bacteria Pseudomonas syringae pv. syringae B728a. Five-week-old N. benthamiana plants were dipped with bacterial solutions of 1×10⁶ cfu/ml and observed 3 days later. Experiments were repeated 3 times with similar results.

### SUMMARY OF THE INVENTION

The present invention provides a method for enhancing resistance to pathogens in plants, comprising over-expression of Arabidopsis thaliana Impaired Oomycete Susceptibility 1 (IOS1) or homologs thereof in plants. The present invention also provides transgenic plants with enhanced resistance to pathogens by over-expression of Arabidopsis thaliana Impaired Oomycete Susceptibility 1 (IOS1) or homologs thereof in the plants.

### DETAILED DESCRIPTION OF THE INVENTION

Plant diseases affect agriculture productivity and economy. In order to resolve the problem, the present invention combines bio-techniques and genetic biology. In the present invention, the applicants discovered that overexpression of Impaired Oomycete Susceptibility 1 (IOS1) in plants primes the pattern-triggered immunity (PTI) response leading to enhanced plant protection against a broad range of pathogens. In addition, the present invention also indicates that IOS1 associates with pattern recognition receptors such as EF-TU RECEPTOR (EFR) and FLAGELLIN SENSING2 (FLS2). Arabidopsis thaliana with a mutation in IOS1 are more susceptible to bacteria, and over-expression of IOS1 increases resistance to microbial pathogens. The applicants also discovered that in IOS1 over-expression lines, the PTI response is not directly induced, but potentiated upon pathogens perception. The present invention demonstrates that over-expression of IOS1 can be used to increase plant resistance to pathogens. On the other hand, the invention also demonstrates that IOS1 shows protective effect when transferred to species other than Arabidopsis thaliana, such as Nicotiana benthamiana.

The terms used in the description herein will have their ordinary and common meaning as understood by those skilled in the art, unless specifically defined otherwise. As used throughout the instant application, the following terms shall have the following meanings:

The term “priming” used in the disclosure refers to that plants after biological or chemical treatment will make more rapid defense response compared to untreated plants under biotic or abiotic stress. This process is known as priming. Of note is that treatment would not induce defense response directly.

The term “Impaired Oomycete Susceptibility 1” or “IOS1” used in the disclosure refers to Arabidopsis thaliana Impaired Oomycete Susceptibility 1 having the amino acid sequence of SEQ ID NO: 2, or a protein encoded by the nucleotide sequence of SEQ ID NO: 1.

The term “homolog(s)” used in the disclosure refers to a gene related to a second gene by descent from a common ancestral DNA sequence. Homolog may apply to the relationship between genes separated by the event of speciation (i.e. ortholog) or to the relationship between genes separated by the event of genetic duplication (i.e. paralog). Orthologs are genes in different species that evolved from a common ancestral gene by speciation. Normally, orthologs retain the same function in the course of evolution. Identification of orthologs is critical for reliable prediction of gene function in newly sequenced genomes. Paralogs are genes related by duplication within a genome. Orthologs retain the same function in the course of evolution, whereas paralogs evolve new functions, even if these are related to the original one. The term “homolog(s)” used in the disclosure mainly refers to homologous sequences with the same or similar function and not limited to orthologous or paralogous.

In the present invention, homologs of Arabidopsis thaliana Impaired Oomycete Susceptibility 1 include but not limited to any protein of which the amino acid sequence has at least 40%, preferably 60%, more preferably 80% and yet more preferably 90% homology with a common amino acid sequence of IOS1. An example is the protein having amino acid sequence of SEQ ID NO: 2.

The term “pathogen” used in the disclosure refers to a microorganism in a wide sense, such as a virus, bacterium, parasite or fungus that causes disease in plants.

The term “over-expression” or “over-expressing” used in the disclosure refers to regulating gene expression of IOS1 to produce more IOS1 than in corresponding wild-type plants under normal conditions. More particularly, in an embodiment of the present invention, a 35S promoter is used to induce IOS1 gene expression persistently, and thus generate more IOS1 than in wild-type plants. Method for over-expressing protein in vivo is well known in the art. Any possible method for over-expressing IOS1 gene or IOS1 protein in plants is included in the present invention, including transcriptional or translational regulation. For example, regulation of gene expression can be used, that is, using appropriate promoter and/or transcriptional or translational enhancer to regulate the performance of the gene itself. Alternatively, regulation of expression as described above can be achieved by an indirect method, for example, due to higher levels and/or activity of the factor of controlling IOS1 gene expression. In an embodiment of the present invention, an enhancer can be used to promote gene transcript levels in gene clusters.

According to the present invention, any promoter or enhancer for improving performance of IOS1 can be used to prepare an expression construct (also known as an expression vector), which is used to introduce and express IOS1 in plants to produce transgenic plants. For example, in an embodiment of the present invention, a 35S promoter with the amino acid sequence of SEQ ID NO: 12 is used to improve IOS1 expression. An expression construct used to over-expressing IOS1 in plants includes, but not limited to, a DNA plasmid named 35S-IOS1-GFP (S1G51800HGF) which is available from Arabidopsis Biological Resource Center (ABRC). The DNA plasmid comprises a 35S promoter, a region encoding IOS1 protein and a green fluorescent protein (GFP), in which the sequence of the 35S promoter and IOS1 is shown as SEQ ID NO: 12 and SEQ ID NO: 1, respectively. GFP is well known to the skilled in the art. The DNA plasmid can be purchased or be prepared by methods well known in the art.

The indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

Thus, the present invention provides a method for enhancing resistance to pathogens in plants, comprising over-expressing Arabidopsis thaliana Impaired Oomycete Susceptibility 1 (IOS1) or homologs thereof in the plants. In an embodiment, an expression construct comprising the nucleotide sequence encoding IOS1 is transformed into the plants to produce transgenic plants over-expressing IOS1. More particularly, the nucleotide sequence encoding IOS1 is SEQ ID NO: 1, and the amino acid sequence of IOS1 is SEQ ID NO: 2. In an embodiment, the pathogens comprise bacteria, fungi, parasites and/or viruses. More particularly, the pathogens comprise Pseudomonas syringae pv. tomato DC3000, Pseudomonas syringae pv. syringae B728a and/or Botrytis cinerea. In an embodiment, the plants are monocotyledonous plants or dicotyledonous plants. More particularly, the plants comprise Solanaceae plants (e.g. tomato, tobacco), Gramineous plants (e.g. rice) and/or Legumes (e.g. soybean).

The present invention also provides a transgenic plant over-expressing Arabidopsis thaliana Impaired Oomycete Susceptibility 1 (IOS1) or homologs thereof, which has enhanced resistance to pathogens. More particularly, the nucleotide sequence encoding IOS1 is SEQ ID NO: 1, and the amino acid sequence of IOS1 is SEQ ID NO: 2. In an embodiment, the enhanced resistance to pathogens means that after infection by pathogens, the following characteristics occurred in the transgenic plant compared to a wild-type plant include (but not limited to): smaller infection area, increased callose (β-1,3 glucan) deposition, enhanced PTI marker gene (such as FRK1) expression, higher MPK3 and MPK6 activities and inhibited pathogen-mediated stomata re-opening to avoid invasion of pathogens. These characteristics may occur individually or simultaneously in any combination. In an embodiment, the pathogens comprise bacteria, fungi, parasites and/or viruses. More particularly, the pathogens comprise Pseudomonas syringae pv. tomato DC3000, Pseudomonas syringae pv. syringae B728a and/or Botrytis cinerea. In an embodiment, the plants are monocotyledonous plants or dicotyledonous plants. More particularly, the plants comprise Solanaceae plants (e.g. tomato, tobacco), Gramineous plants (e.g. rice) and/or Legumes (e.g. soybean).

### EXAMPLES

The examples below are non-limiting and are merely representative of various aspects and features of the present invention.

### Example 1

### Materials and Methods

### Preparation of IOS1 Over-Expression Arabidopsis Plants

The DNA plasmids (35S-IOS1-GFP) expressing IOS1 protein fused with GFP at the C terminus under the control of the cauliflower mosaic virus 35S promoter were obtained from Arabidopsis Biological Resource Center (ABRC) (ABRC stock S1G51800HGF). The expression construct (vector) was as described by Gou et al. (Gou, X. P., He, K., Yang, H., Yuan, T., Lin, H. H., Clouse, S. D., and Li, J. (2010). Genome-wide cloning and sequence analysis of leucine-rich repeat receptor-like protein kinase genes in Arabidopsis thaliana. BMC Genomics 11). The DNA plasmids were transformed into Agrobacterium tumefaciens strain GV3101 by electroporation, followed by antibiotic screening (Rifampicin+Kanamycine+Gentamycin). Agrobacterium tumefaciens carrying 35S-IOS1-GFP were quantitatively determined. Flowers of Arabidopsis thaliana wild-type were dipped in suspension of transformed Agrobacterium tumefaciens for infection. Plants with successful transformation were determined by screening on 1.5% MS agar plates containing 50 μM glufosinate-ammonium (Fluka) and raised to homozygous T3 lines for subsequent experiments.

Generation of Nicotiana benthamiana Transgenics with IOS1 Over-Expression

To create transgenic lines, seeds of N. benthamiana were surface sterilized by incubation in 20% of household bleach for 10 min followed by 5 washes with sterile distilled water. The surface sterilized seeds were sowed on MS medium (M0222, Duchefa Biochemie) plates containing 1% sucrose and grown for one month. Colonies of A. tumefaciens strain LBA4404 carrying the DNA plasmids 35S-IOS1-GFP were grown into 50 mL liquid YEP medium (10 g/L yeast extract, 10 g/L bacto peptone, 5 g/L NaCl, pH 7.0) containing 100 mg/L rifampicin and 50 mg/L kanamycin at 28° C. with constant shaking (340 rpm) overnight. The cells were harvested by centrifugation at 4000 g for 10 min and the bacterial pellet was suspended to a final OD₆₀₀ of 0.6 to 0.8 in co-culture medium (MS with vitamins, 3% sucrose, 0.1 mg/L 1-naphthaleneacetic acid, 1 mg/L benzoic acid, 100 uM acetosyringone). For plant transformation, the upper leaves of sterile N. benthamiana were excised and cut into pieces of 1 cm² in area by avoiding leaf margins and mid ribs. The explants were immersed in the prepared Agrobacterium inoculum on a Petri plate for 1 minute. After inoculation, the explants were blotted dried on sterile tissue paper and placed, the adaxial side up, on to co-culture plates (co-culture medium containing 0.2% phytagel). The explants were co-cultivated in the dark for 2 days. After co-cultivation, the explants were transferred to shooting medium (MS with vitamins, 3% sucrose, 0.1 mg/L 1-naphthaleneacetic acid, 1 mg/L benzoic acid, 200 mg/L timentin, 15 mg/L hygromycin, 0.2% phytagel) for transformant selection. The regenerating explants were transferred to fresh shooting medium every 2 weeks. Shoots were excised once they reached a length of 5-10 mm and transferred to rooting medium (MS with vitamins, 3% sucrose, 200 mg/L timentin, 0.2% phytagel). Two single insertion lines per construct were further screened for homozygous progenies using segregation analyses of hygromycin resistance. Homozygous T3 plants were used for the assays presented.

### Pathogens Infection Assays

Bacteria Infection in Arabidopsis thaliana:

Five-week-old Arabidopsis thaliana transgenic plants were dipped in a suspension of 1×10⁶ cfu/ml Pseudomonas syringae pv. tomato DC3000 (Pst DC3000) for 15 mins After inoculation, plants were kept at 100% relative humidity, and symptoms were evaluated 3 days later. Bacterial titers were determined as previously described for Pst DC3000 (Zimmerli, L., Jakab, C., Metraux, J. P., and Mauch-Mani, B. (2000). Potentiation of pathogen-specific defense mechanisms in Arabidopsis by beta-aminobutyric acid. P Natl Acad Sci USA 97, 12920-12925).

Bacteria Infection in Nicotiana benthamiana:

Bacterial strain Pseudomonas syringae pv. syringae B728a was a kind gift from Dr. Nai-Chun Lin (Department of Agricultural Chemistry, National Taiwan University). Five-week-old N. benthamiana plants were dipped in bacterial solutions of 1×10⁶ cfu/mL Pss B728a for 5 min. The infected plants were kept at 100% relative humidity for 1 day, and symptoms were evaluated 3 days later (i.e. 4 days after inoculation).

### Fungus:

The fungus Botrytis cinerea was obtained from C. Y. Chen (Taiwan University, Taipei, Taiwan). Five Arabidopsis thaliana five-week-old transgenic plants were selected, and three leaves with the same size were selected from each plant for drop-inoculation. Droplets of 10 μl of Botrytis cinerea suspension (1×10⁵ spores/mL) were deposited on the selected leaves. Plants were kept at 100% relative humidity, and symptoms were evaluated 3 days later.

Callose (β-1,3 glucan) Deposition Assay

Leaves of five-week-old Arabidopsis thaliana transgenic plants were syringe infiltrated with 1 μM flg22 (the amino acid sequence is QRLSTGSRINSAKDDAAGLQIA (SEQ ID NO: 3)) in 10 mM MgSO₄. flg22 is a 22-amino acid derived peptide from the microbe- or pathogen-associated molecular pattern flagellin, which is known to be recognized by the PRR flagellin-sensitive-2 (FLS2). Control plants were infiltrated with 10 mM MgSO4 buffer only. Nine leaf discs from each experiment group were selected for analyses. Harvested leaf samples were cleared overnight by incubation in 95% ethanol at room temperature and then washed three times (2 h for each washing) with sterilized water. Cleared leaves were stained with 0.01% aniline blue in 0.15 M phosphate buffer, pH 9.5, for 24 h. Callose deposits were visualized under UV illumination using a Nikon Optiphot-2 microscope. Callose deposits were counted using the “analyze particles” function of ImageJ (http://rsb.info.nih.gov/ij/) (Singh P., Kuo Y. C., Mishra S., Tsai C. H., Chien C. C., Chen C. W., et al. (2012). The lectin receptor kinase-VI.2 is required for priming and positively regulates Arabidopsis pattern-triggered immunity. Plant Cell 24 1256-1270).

### Gene Expression Analysis

For FRK1 expression analyses in IOS1 over-expression lines, 5-week-old Arabidopsis thaliana transgenic plants were syringe-infiltrated with 10 mM MgSO₄ (Mock) or 1×10⁸ cfu/ml Pst DC3000 hrcC (mutants of Pst DC3000) and samples were collected 90 min later. Total RNA isolation, complementary DNA biosynthesis and real-time PCR analyses were performed according to Wu et al (Wu C. C., Singh P., Chen M. C., Zimmerli L. (2010). L-Glutamine inhibits beta-aminobutyric acid-induced stress resistance and priming in Arabidopsis. J. Exp. Bot. 61: 995-1002). Normalization of gene expression across different samples was performed with EF-1 and UBQ10 as internal controls. For RT-PCR, one microliter of cDNA was used as template and standard PCR conditions were applied as described (Singh P., Kuo Y. C., Mishra S., Tsai C. H., Chien C. C., Chen C. W., et al. (2012). The lectin receptor kinase-VI.2 is required for priming and positively regulates Arabidopsis pattern-triggered immunity. Plant Cell 24 1256-1270). Primers used were shown in Table 1.

TABLE 1

| | Forward | Reverse |
|---|---|---|
| Gene | primer | primer |
| IOS1 (NCBI gene | 5′-CTTGACCGGAGAGATCTTAG-3′ | 5′-AGCTAGAGAAACTCTGGGACTG-3′ |
| bank No: | (SEQ ID NO: 4) | (SEQ ID NO: 5) |
| At1g51800) | | |
| FRK1 (NCBI gene | 5′-GCCAACGGAGACATTAGAG-3′ | 5′-CCATAACGACCTGACTCATC-3′ |
| bank No: | (SEQ ID NO: 6) | (SEQ ID NO: 7) |
| At2g19190) | | |
| EF-1 (NCBI gene | 5′-TGAGCACGCTCTTCTTGCTTTCA-3′ | 5′-GGTGGTGGCATCCATCTTGTTACA-3′ |
| bank No: | (SEQ ID NO: 8) | (SEQ ID NO: 9) |
| At5g60390) | | |
| UBQ10 (NCBI gene | 5′-GGCCTTGTATAATCCCTGATGA-3′ | 5′-AAAGAGATAACAGGAACGGAAA-3′ |
| bank No: | (SEQ ID NO: 10) | (SEQ ID NO: 11) |
| At4g05320) | | |

### MAP Kinase Assay

Leaves of 5-week-old Arabidopsis thaliana transgenic plants were syringe-infiltrated with 1 nM flg22 or 10 mM MgSO4 (control) for 5 min before being pooled for harvest. MAP kinase assays were performed as described (Singh P., Kuo Y. C., Mishra S., Tsai C. H., Chien C. C., Chen C. W., et al. (2012). The lectin receptor kinase-VI.2 is required for priming and positively regulates Arabidopsis pattern-triggered immunity. Plant Cell 24 1256-1270). The signals were visualized using an enhanced chemiluminescence system (Western Lightning Plus-ECL kit; Perkin-Elmer) following the manufacturer's instructions.

### Stomatal Assay

This assay was performed according to steps disclosed by Melotto et al. (Melotto, M., Underwood, W., Koczan, J., Nomura, K., and He, S. Y. (2006). Plant stomata function in innate immunity against bacterial invasion. Cell 126, 969-980). The lower epidermis of leaves of five-week-old Arabidopsis thaliana transgenic plants was removed and put into MES buffer (10 mM MES-KOH, 30 mM Kcl, pH 6.15) for at least 3 hours to open stomata. 1×10⁸ cfu/ml Pst DC3000 were added into the buffer. The observation was performed at indicated time by Olympus BX51 microscope. The width of the stomatal aperture was measured using the “measure” function of ImageJ (http://rsb.info.nih.gov/ij/).

### Results

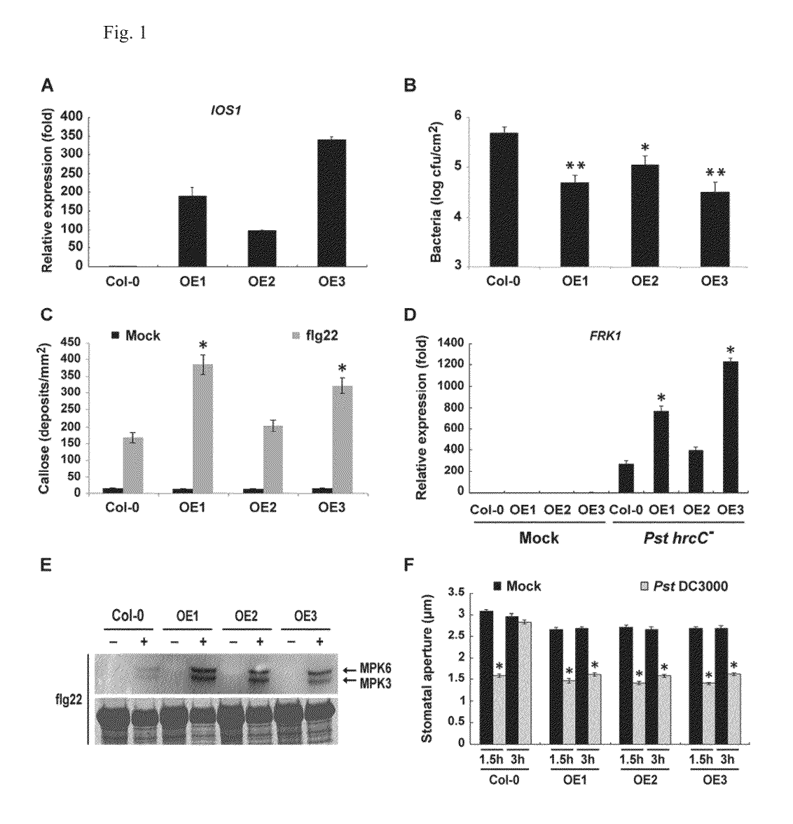

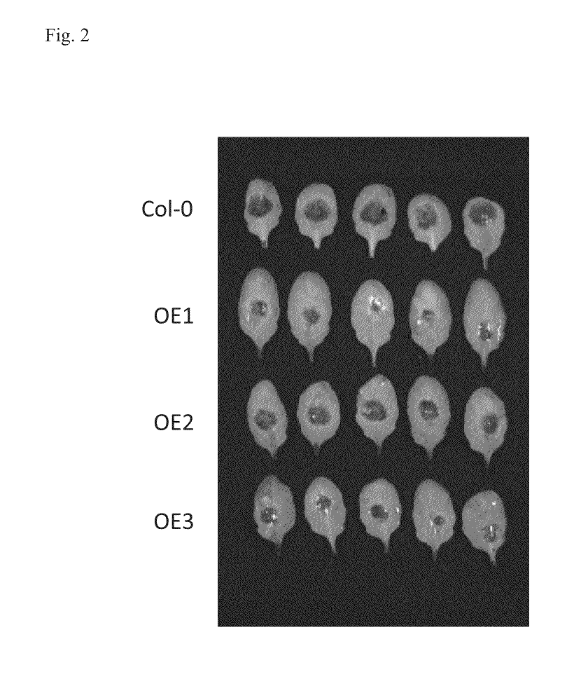

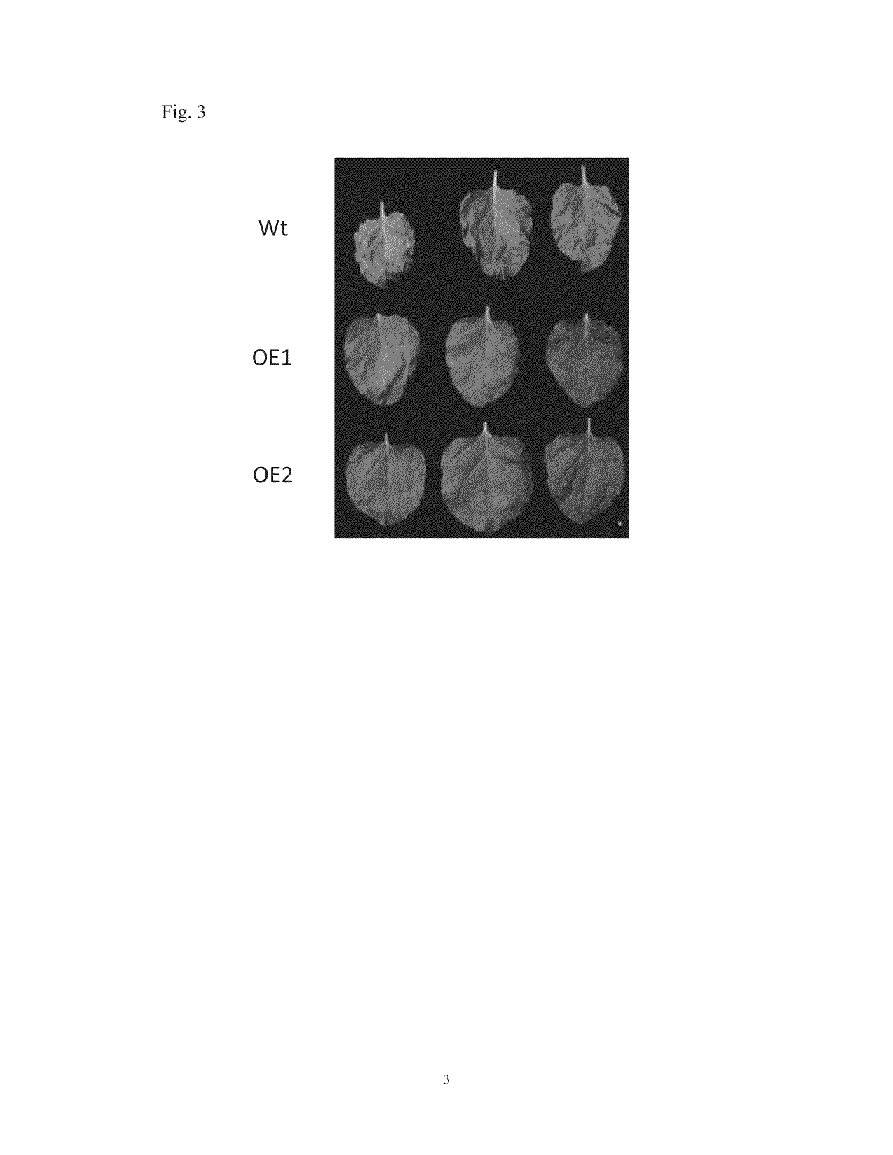

Arabidopsis thaliana transgenic plants over-expressing IOS1 (OE1, OE2 and OE3) demonstrated increased resistance to Pst DC3000 bacteria (FIG. 1B) and to the fungus B. cinerea (FIG. 2). FIG. 1C shows that OE1, OE2 and OE3 did not directly induce constitutive callose deposition, while significantly more callose deposition was observed in over-expression lines after elicitation with the MAMP flg22. FIG. 1D shows that OE1, OE2 and OE3 did not induce constitutive FRK1 gene expression, while significantly more FRK1 gene expression was observed after pathogens inoculation. FRK1 is a PTI-responsive gene as described in Singh et al (Singh P., Kuo Y. C., Mishra S., Tsai C. H., Chien C. C., Chen C. W., et al. (2012). The lectin receptor kinase-VI.2 is required for priming and positively regulates Arabidopsis pattern-triggered immunity. Plant Cell 24 1256-1270). In FIG. 1E, MPK3 and MPK6 activities were higher in IOS1 over-expression transgenic lines upon elicitation with PAMPs. In FIG. 1F, over-expression of IOS1 inhibited the bacteria-mediated re-opening of stomata in transgenic plants OE1, OE2 and OE3 after 3 hours of bacterial exposure, thus limiting invasion of bacteria. In summary, transgenic plants over-expressing IOS1 are more resistant to microbial pathogens through a stronger activation of the general and broad spectrum resistance known as PTI.

FIG. 3 showed that Nicotiana benthamiana transgenic plants over-expressing IOS1 (OE1 and OE2) were more resistant to bacteria. When wild-type (Wt) plants exhibited severe disease symptoms with spreading lesions necrosis, transgenic lines harboring the 35S-IOS1-GFP constructs developed less disease symptoms. This result verified that the protective effect of IOS1 could work in not only Arabidopsis thaliana but also in other plant species.

One skilled in the art readily appreciates that the present invention is well adapted to carry out the objects and obtain the ends and advantages mentioned, as well as those inherent therein. The transgenic plants, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for enhancing resistance to pathogens in plants, comprising over-expressing Arabidopsis thaliana Impaired Oomycete Susceptibility 1 (IOS1) or homologs thereof in the plants.

2. The method of claim 1, wherein an expression construct comprising the nucleotide sequence encoding IOS1 is transformed into the plants to produce transgenic plants over-expressing IOS1.

3. The method of claim 2, wherein the nucleotide sequence encoding IOS1 is SEQ ID NO: 1.

4. The method of claim 1, wherein the pathogens comprise Pseudomonas syringae pv. tomato DC3000, Pseudomonas syringae pv. syringae B728a and/or Botrytis cinerea.

5. The method of claim 1, wherein the plants comprise Solanaceae plants, Gramineous plants and/or Legumes.

6. A transgenic plant over-expressing Arabidopsis thaliana Impaired Oomycete Susceptibility 1 (IOS1) or homologs thereof, which has enhanced resistance to pathogens.

7. The transgenic plant of claim 6, wherein the amino acid sequence of IOS1 is SEQ ID NO: 2.

8. The transgenic plant of claim 6, wherein after infection by pathogens, the transgenic plant comprises characteristics selected from the group consisting of: smaller infection area, increased callose (β-1,3 glucan) deposition, enhanced PTI marker genes such as FRK1 gene expression, higher MPK3 and MPK6 activities and inhibited pathogen-mediated stomata re-opening, when compared to the wild-type control plants.

9. The transgenic plant of claim 6, wherein the pathogens comprise Pseudomonas syringae pv. tomato DC3000, Pseudomonas syringae pv. syringae B728a and/or Botrytis cinerea.

10. The transgenic plant of claim 6, wherein the plant is selected from Solanaceae plants, Gramineous plants and Legumes.


SEQUENCE LISTING (12 sequences)

SEQ ID NO: 1 - DNA, 2685 bp, Arabidopsis thaliana
  CDS (1)..(2685)
atggcgttttcttcttgttttctcttagttcttctgcaaatcttctcagctttacttctttgtcttgctcaagatcagtcaggattcatcagtttggattgcggttcgcctagagaaactagctttcgtgagaaaacaacaaacattacttacatttccgatgcgaatttcatcaataccggtgttggtggaagcatcaaacaaggataccggactcagtttcagcaacaaacatggaacctacggagttttccacaaggcataagaaactgctataccttaaacctaaccataggcgacgaatatctaatcagagccaatttccttcacggcggttatgacgacaaaccctcaacgcaattcgagctctacctcggacctaatctttggtctacagttacaaccacgaacgagaccgaagcatcaatctttgagatgattcatatcttgaccactgatcgtttacaaatctgtctggtcaagacaggaaacgcaacgccttttatctccgctttagagcttcgtaaactcatgaacacaacttacttgactcgacaaggttccttgcagaccttcatcagagcagacgttggcgcgactgttaaccaaggctacaggtacggaatcgatgtgtttgatcgtgtctggactccgtataatttcggaaactggtcacagattagtaccaatcagtcagtgaatataaacaatgactatcaaccaccagagattgctatggtcacagcctctgttccaacagacccggacgcagctatgaacatttcgctagtcggggtggaacgtactgtgcagttctacgtttttatgcattttgcagagattcaagagctcaaatccaatgatacaagagagttcaacatcatgtacaataacaagcatatctatggaccttttagaccacttaatttcaccacgtcttccgtatttacacctaccgaagttgtcgccgatgcaaatgggcagtacatattttcgcttcaaagaacaggaaattcgactttacctcctctgctcaacgctatggagatttattcggttaacttgttgccgcaacaggaaactgatagaaaagaagttgatgcaatgatgaacatcaagtcggcttacggcgtgaacaagattgactgggaaggagatccttgcgtgccgctggattacaagtggtctggtgttaattgcacttatgttgacaatgagaccccaaagatcatttctttggatctgtctacaagtggcttgaccggagagatcttagaatttatatcagaccttaccagtttagaagttcttgacttgtctaacaattctttaactggttcagtcccagagtttctagctaatatggaaaccttaaaactcataaatctatctggaaatgagctaaatggctctatccctgcaactctccttgataaagaacgacgaggatctattacgttaagtatcgaaggaaataccgggctttgttcatctacatcatgcgctaccacgaaaaagaagaagaaaaacactgttattgcacctgttgcagcatcacttgtttcagtattcctcattggagctggaatcgttactttccttatcctcaagaggaagaagagaaccaagcttggtttgaatcctaattcaggaactggtacaacaccgttacattcgaggtctcaccacggttttgaacctcccgttatagcgaaaaaccggaaattgacttacattgatgttgttaagatcacaaacaactttgagagggttcttggtaggggtggttttggtgtggtttattacggcgtcttgaataatgaaccagttgcggtaaagatgctcactgagtccactgcacttggttacaaacagtttaaagccgaggttgagttgcttcttagagttcatcacaaagatctcacatgtcttgttggatattgtgaagaaggagataaaatgtctctaatctatgagttcatggcgaatggtgacttgaaagagcacttatcagggaagcgtggaccgtcgatactaacttgggaaggaaggcttcgtatagcagcggaatcagcgcaaggattggaatatttgcacaatggatgcaaaccgcaaatagttcaccgcgacattaagactactaacatcttattaaacgagaaatttcaagctaagcttgcggatttcgggctttcacggtcttttccacttggaactgaaactcacgtctcaaccatagttgctggaactcccggatatcttgatcctgaatactacagaactaattggttgaccgagaaaagtgatgtgttcagctttggtgttgttctactagagctagtaacaaaccaacccgtgatagacatgaaaagagaaaaatcacacatagctgaatgggtaggtttgatgctatcaagaggagacattaacagcattgtagatcctaagcttcaaggagactttgatccaaacacgatatggaaagttgttgagaccgcaatgacttgcctgaatccgtcttcttcacggagaccgacaatgactcaagtcgtaatggatctgaaagaatgtctgaacatggagatggcaagaaacatgggaagccgtatgacggattcaactaatgattcttcaattgagctgtccatgaatttcacaacagagcttaatccaggagctagatga

SEQ ID NO: 2 - protein, 894 aa, Arabidopsis thaliana
MAFSSCFLLVLLQIFSALLLCLAQDQSGFISLDCGSPRETSFREKTTNITYISDANFINTGVGGSIKQGYRTQFQQQTWNLRSFPQGIRNCYTLNLTIGDEYLIRANFLHGGYDDKPSTQFELYLGPNLWSTVTTTNETEASIFEMIHILTTDRLQICLVKTGNATPFISALELRKLMNTTYLTRQGSLQTFIRADVGATVNQGYRYGIDVFDRVWTPYNFGNWSQISTNQSVNINNDYQPPEIAMVTASVPTDPDAAMNISLVGVERTVQFYVFMHFAEIQELKSNDTREFNIMYNNKHIYGPFRPLNFTTSSVFTPTEVVADANGQYIFSLQRTGNSTLPPLLNAMEIYSVNLLPQQETDRKEVDAMMNIKSAYGVNKIDWEGDPCVPLDYKWSGVNCTYVDNETPKIISLDLSTSGLTGEILEFISDLTSLEVLDLSNNSLTGSVPEFLANMETLKLINLSGNELNGSIPATLLDKERRGSITLSIEGNTGLCSSTSCATTKKKKKNTVIAPVAASLVSVFLIGAGIVTFLILKRKKRTKLGLNPNSGTGTTPLHSRSHHGFEPPVIAKNRKLTYIDVVKITNNFERVLGRGGFGVVYYGVLNNEPVAVKMLTESTALGYKQFKAEVELLLRVHHKDLTCLVGYCEEGDKMSLIYEFMANGDLKEHLSGKRGPSILTWEGRLRIAAESAQGLEYLHNGCKPQIVHRDIKTTNILLNEKFQAKLADFGLSRSFPLGTETHVSTIVAGTPGYLDPEYYRTNWLTEKSDVFSFGVVLLELVTNQPVIDMKREKSHIAEWVGLMLSRGDINSIVDPKLQGDFDPNTIWKVVETAMTCLNPSSSRRPTMTQVVMDLKECLNMEMARNMGSRMTDSTNDSSIELSMNFTTELNPGAR

SEQ ID NO: 3 - protein, 22 aa, Artificial Sequence
  Synthetic flg22 peptide
QRLSTGSRINSAKDDAAGLQIA

SEQ ID NO: 4 - DNA, 20 bp, Artificial Sequence
  Forward primer for IOS1in PCR
cttgaccggagagatcttag

SEQ ID NO: 5 - DNA, 22 bp, Artificial Sequence
  Reverse primer for IOS1 in PCR
agctagagaaactctgggactg

SEQ ID NO: 6 - DNA, 19 bp, Artificial Sequence
  Forward primer for FRK1 in PCR
gccaacggagacattagag

SEQ ID NO: 7 - DNA, 20 bp, Artificial Sequence
  Reverse primer for FRK1 in PCR
ccataacgacctgactcatc

SEQ ID NO: 8 - DNA, 23 bp, Artificial Sequence
  Forward primer for EF-1 in PCR
tgagcacgctcttcttgctttca

SEQ ID NO: 9 - DNA, 24 bp, Artificial Sequence
  Reverse primer for EF-1 in PCR
ggtggtggcatccatcttgttaca

SEQ ID NO: 10 - DNA, 22 bp, Artificial Sequence
  Forward primer for UBQ10 in PCR
ggccttgtataatccctgatga

SEQ ID NO: 11 - DNA, 22 bp, Artificial Sequence
  Reverse primer for UBQ10 in PCR
aaagagataacaggaacggaaa

SEQ ID NO: 12 - DNA, 1332 bp, Cauliflower mosaic virus
  promoter (1)..(1332)
tccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacac